(19) United States
(12) Patent Application Publication
(10) Pub. No.: US 2019/0125561 A1
(43) Pub. Date: May 2, 2019

(54) SYSTEM AND METHOD FOR DEPLOYING A STENT IN A VESSEL OF A SUBJECT

(71) Applicants: Robert M. SULLIVAN, Orlando, FL (US); Sean S. AHN, Winter Garden, FL (US)

(72) Inventors: Robert M. SULLIVAN, Orlando, FL (US); Sean S. AHN, Winter Garden, FL (US)

(21) Appl. No.: 16/141,300

(22) Filed: Sep. 25, 2018

Related U.S. Application Data
(60) Provisional application No. 62/579,069, filed on Oct. 30, 2017.

(51) Int. Cl. A61F 2/962; A61F 2/958; A61F 2/82

(52) U.S. Cl. CPC A61F 2/962; A61F 2002/9522; A61F 2/82; A61F 2/958

(57) ABSTRACT

A method and assembly are provided for securing a stent within a vessel of a subject. The method includes moving the stent to a position along the vessel and inflating a balloon positioned on a downstream side of the stent. The method further includes deploying the stent at the position and verifying that the deployed stent does not float from the position in a downstream direction relative to the inflated balloon. The method further includes deflating the balloon. The assembly includes the stent, a plurality of channels internal to the stent and a deployment mechanism configured to deploy the stent when the stent is positioned in the vessel of the subject. In another embodiment, a system is provided that includes the assembly, a guide wire, a balloon lumen, an inlet port to inject fluid into the balloon lumen and a handle operatively coupled to the deployment mechanism.

### BACKGROUND

Vessel stenosis is an abnormal narrowing in a blood vessel or other tubular organ or structure. Conventional treatment of vessel stenosis involves deployment of a stent within the blood vessel to a position with a narrowed diameter. The stent is moved along a guide wire to this position and is then deployed so that the stent permanently applies pressure to the vessel walls to keep the blood vessel open. After deploying the stent, a balloon is moved along the guide wire within the deployed stent and is inflated to secure the stent against the vessel walls and widen the diameter of the vessel before the balloon is deflated and removed from the vessel.

### SUMMARY

The current inventor recognized that conventional stent deployment systems have notable drawbacks. For example, in the event that the deployment diameter of the stent is less than an inner diameter of the vessel opening, the deployed stent may not secure to the vessel opening and thus may float along the blood vessel. This presents significant health risks to the subject, such as the stent floating along the blood vessel to the heart and potentially requiring open heart surgery to retrieve the stent. The inventor of the present invention noticed that conventional stent systems fail to provide a safety measure to prevent the deployed stent from floating along the blood vessel. Although conventional stent systems provide a balloon that is moved within the deployed stent and inflated to secure the deployed stent against the interior of the blood vessel, no safety measure is employed between the time that the stent is deployed and the time that the balloon is inflated within the deployed stent. Thus, the inventor of the present invention noticed that there is a safety risk to the subject during this time gap when the deployed stent may float along the blood vessel. The method and assembly described herein is provided to address this safety risk.

In a first set of embodiments, an assembly is provided for deploying a stent in a vessel of a subject. The assembly includes the stent and a plurality of channels positioned internal to the stent. The assembly also includes a deployment mechanism configured to deploy the stent when the stent is positioned in the vessel of the subject.

In a second set of embodiments, a method is provided for deploying the stent in the vessel of the subject. The method includes moving the stent to a position along the vessel. The method also includes inflating a balloon positioned on a downstream side of the stent. The method further includes deploying the stent at the position along the vessel and verifying that the deployed stent does not float from the position in a downstream direction relative to the inflated balloon. The method also includes deflating the balloon.

In a third set of embodiments, a system is provided for deploying the stent in the vessel of the subject. The system includes a guide wire, a balloon lumen, a stent and a plurality of channels positioned internal to the stent including a first channel to receive the guide wire and a second channel to receive the balloon lumen. The system also includes a deployment mechanism configured to deploy the stent when the stent is positioned in a vessel of the subject. The system further includes a balloon positioned on a downstream side of the stent. The system further includes an inlet port at an upstream end of the second channel that is configured to direct injected fluid into the balloon lumen. The system further includes a handle operatively coupled to the deployment mechanism such that movement of the handle is configured to deploy the stent.

In a fourth set of embodiments, a balloon assembly is provided for a stent device, where the stent device includes a guide wire, a stent, a plurality of channels internal to the stent including a first channel to receive the guide wire and a deployment mechanism configured to deploy the stent when the stent is positioned in a vessel of a subject. The balloon assembly includes a balloon lumen that is received within a second channel of the plurality of channels and a balloon positioned on a downstream side of the stent, where the first channel passes through an opening formed by the balloon and where the second channel extends within an interior of the balloon such that fluid injected into the balloon lumen is configured to inflate the balloon.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1A is a cross-sectional view of an assembly for deploying a stent in a vessel of a subject, according to an embodiment;

FIG. 1B is a cross-sectional view of the assembly of FIG. 1A taken along the line 1B-1B, according to an embodiment;

FIG. 1C is a cross-sectional view of the assembly of FIG. 1A taken along the line 1C-1C, according to an embodiment;

FIG. 1D is a top view that illustrates example components of a system including the assembly of FIG. 1A, according to an embodiment;

FIG. 1E is a top view that illustrates example components of the assembly of the system of FIG. 1D, according to an embodiment;

FIG. 2 is a flow chart that illustrates an example method for deploying the stent in a vessel of a subject, according to an embodiment;

FIG. 3A is a side view that illustrates example components of the assembly of FIG. 1E where the stent is positioned at a position in a vessel with a narrow opening, according to an embodiment;

FIG. 3B is a side view that illustrates example components of the assembly of FIG. 1E where the balloon positioned on a downstream side of the stent is inflated, according to an embodiment;

FIG. 3C is a side view that illustrates example components of the assembly of FIG. 1E where the deployment mechanism is activated to deploy the stent, according to an embodiment;

FIG. 3D is a side view that illustrates example components of the assembly of FIG. 1E where the deployed stent maintains a minimum gap separation from the inflated balloon, according to an embodiment;

FIG. 3E is a side view that illustrates example components of the assembly of FIG. 1E where the balloon positioned on a downstream side of the stent is deflated, according to an embodiment;

FIG. 3F is a side view that illustrates example components of the assembly of FIG. 1E where the deflated balloon is retracted from the vessel along the guide wire, according to an embodiment;

FIG. 3G is a side view that illustrates example components of the assembly of FIG. 1E where the deflated balloon has been retracted from the vessel, according to an embodiment;

FIG. 3H is a side view that illustrates example components of the assembly of FIG. 1E where a secondary balloon is moved along the guide wire to the position in the vessel within the deployed stent, according to an embodiment;

FIG. 3I is a side view that illustrates example components of the assembly of FIG. 1E where the secondary balloon is inflated to secure the deployed stent along an interior of the vessel, according to an embodiment;

FIG. 3J is a side view that illustrates example components of the assembly of FIG. 1E where the secondary balloon is deflated, according to an embodiment;

FIG. 3K is a side view that illustrates example components of the assembly of FIG. 1E where the secondary balloon is retracted along the guide wire from the vessel, according to an embodiment;

FIG. 4A is a side view that illustrates example components of the assembly of FIG. 1E where the deployed stent maintains a minimum gap separation from a deployed basket, according to an embodiment;

FIG. 4B is a side view that illustrates example components of the assembly of FIG. 1E where the deployed stent maintains a minimum gap separation from a deployed umbrella, according to an embodiment.

### DETAILED DESCRIPTION

A method and assembly are described for deploying a stent in a vessel of a subject. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5× to 2×, for example, about 100 implies a value in a range from 50 to 200. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

Some embodiments of the invention are described below in the context of deploying a stent. In one example embodiment, the invention is described in the context of deploying the stent in a vessel or other tubular organ or structure of a subject. In another example embodiment, the invention is described in the context of deploying the stent in an artery or vein of a subject. In one example embodiment, the invention is described in the context of deploying the stent in the vessel of the subject to treat a condition (e.g. stenosis) in the subject. However, the invention is not limited to this context. In some embodiments, the invention is described in the context of deploying a stent in a venous or arterial vascular tree.

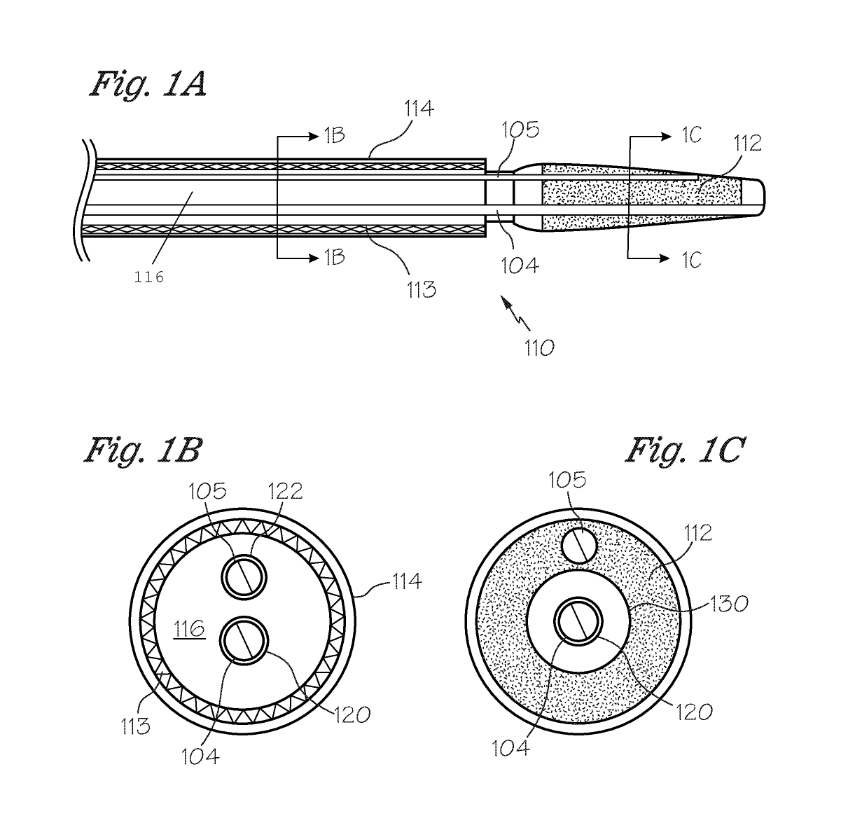

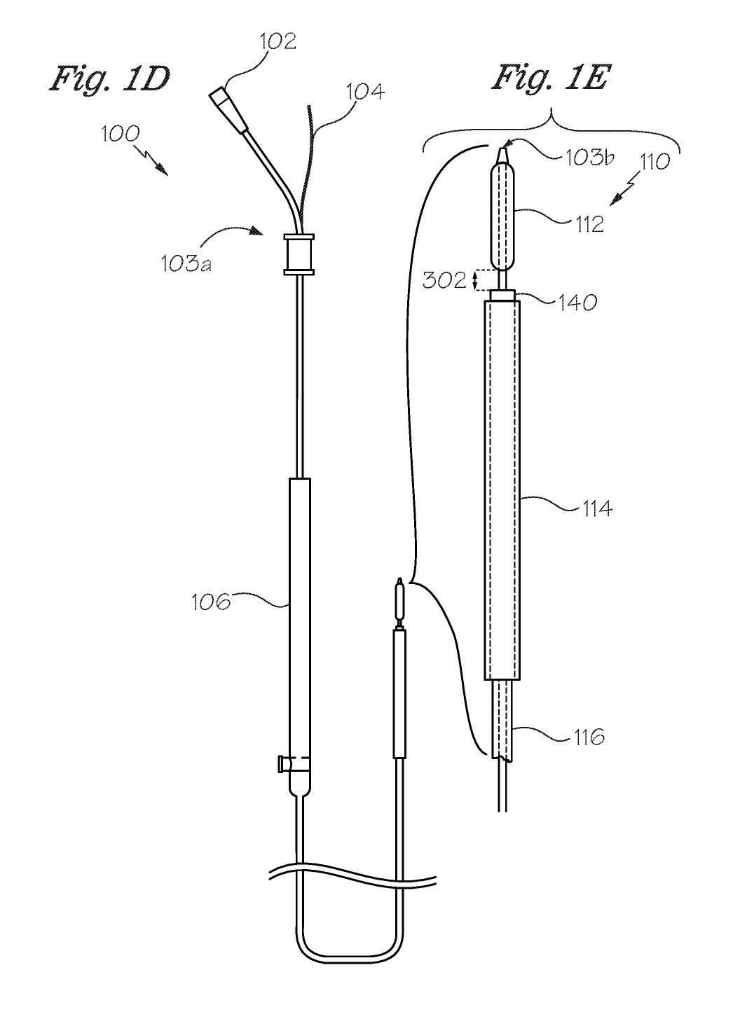

FIG. 1A is a cross-sectional view of an assembly 110 for deploying a stent 113 in a vessel of a subject, according to an embodiment. FIG. 1B is a cross-sectional view of the assembly 110 of FIG. 1A taken along the line 1B-1B, according to an embodiment. FIG. 1C is a cross-sectional view of the assembly 110 of FIG. 1A taken along the line 1C-1C, according to an embodiment. In some embodiments, the stent 113 is a covered stent. In an example embodiment, the covered stent is Gore® Viabahn®, Flagstaff, Ariz. In other embodiments, the stent 113 is a non-covered stent. In an example embodiment, the non-covered stent is E-Luminexx® by Bard®, Tempe Ariz. In still other embodiments, the stent 113 is a drug eluting stent. In an example embodiment, the covered stent is Gore® Viabahn® with Heparin Bioactive Surface, Flagstaff, Ariz. In still other embodiments, the stent 113 is a non-drug eluting stent. In an example embodiment, the covered stent is Gore® Viabahn®, Flagstaff, Ariz. or E-Luminexx® by Bard®, Tempe Ariz.

In an embodiment, the assembly 110 includes a plurality of channels positioned internal to the stent 113. In one embodiment, a catheter 116 positioned within the stent 113 defines a pair of channels 120, 122. In an embodiment, a first channel 120 is configured to receive a guide wire 104 and a second channel 122 is configured to receive a balloon lumen 105. In some instances, the phrase “configured to receive a guide wire” may mean that the channel allows passage of a guide wire therethrough. The phrase “configured to receive a balloon lumen” may mean enclosing the balloon lumen, or allowing passage of a balloon lumen. The balloon lumen 105 may be a separate structure to the second channel 122, or integral to the second channel 122. In another embodiment, the catheter 116 includes a single channel (e.g. channel 120 without channel 122 or a single channel with a diameter larger than channel 120 or 122) and the guide wire 104 and balloon lumen 105 are received within the single channel. In an example embodiment, an inner diameter of the first channel 120 is about 0.035″ or in a range from about 0.02″ to about 0.05″. In an example embodiment, an inner diameter of the second channel 122 is about 0.03″ or in a range from about 0.02″ to about 0.04″. In another example embodiment, the inner diameter of the second channel 122 is in a range from about 0.0001″ to about 0.05″. Although two channels are depicted in FIG. 1B, the assembly 110 is not limited to two channels and may include less than two channels or more than two channels.

In an embodiment, the assembly 110 includes a deployment mechanism that is configured to deploy the stent 113 when the stent 113 is positioned in the vessel of the subject. In one embodiment, the deployment mechanism is a deployment sleeve 114 that encloses the stent 113 and deploys the stent 113 when the deployment sleeve 114 is retracted relative to the stent 113. In another embodiment, the deployment mechanism includes webbing that encloses the stent 113 and a string that unravels the webbing and deploys the stent 113 when the string is pulled. In an example embodiment, a stent 113 that uses such a string deployment mechanism is Gore® Viabahn®, Flagstaff, Ariz.

In an embodiment, the assembly 110 includes a safety mechanism positioned distally to the stent 113 and is configured to obstruct motion of the deployed stent 113 from migrating in the vessel of the subject. In one embodiment, the safety mechanism is a balloon 112 spaced apart from a downstream end 140 of the stent 113. In another embodiment, the balloon 112 is positioned on a downstream side of the stent 113, i.e. positioned downstream of the end 140 of the stent 113. For purposes of this description, “downstream” means a direction that the stent 113 would float along the vessel 301 if the stent 113 was not secured to the vessel 301 wall or a natural direction of fluid flow within the vessel 301. In another embodiment, “downstream” means a direction of fluid flow (e.g. blood flow) in the vessel 301. In an example embodiment, the balloon 112 is a compliant balloon that is configured to accommodate a shape of a vessel passage where it is deployed. In an example embodiment, the balloon 112 has a maximum pressure of about 8 atmospheres (atm) or in a range from about 2 atm to about 50 atm. In another example embodiment, the balloon 112 is made from Kevlar® material. However, the balloon 112 can be made from any material that is resilient to contact with the deployed stent 113. In another embodiment, the balloon 112 is a non-compliant balloon. In an example embodiment, the non-compliant balloon has a maximum pressure of about 20 atmospheres (atm) or in a range from about 2 atm to about 50 atm.

In an embodiment, the balloon 112 forms an opening 130 where the first channel 120 and guide wire 104 pass through. Although not depicted in FIG. 1A, in one embodiment, the first channel 120 and guide wire 104 passes through the opening 130 and beyond the balloon 112. As depicted in FIG. 1C, in one embodiment, the second channel 122 extends within an interior of the balloon 112 such that a downstream end of the second channel 122 and balloon lumen 105 are positioned within the interior of the balloon 112. Based on this arrangement, fluid that is injected into the balloon lumen 105 passes into the interior of the balloon 112 to inflate the balloon 112.

FIG. 1D is a top view that illustrates example components of a system 100 for deploying the stent 113 in a vessel of a subject that includes the assembly 110 of FIG. 1A, according to an embodiment. FIG. 1E is a top view that illustrates example components of the assembly 110 of FIG. 1A, according to an embodiment. The assembly 110 includes an upstream end 103a and a downstream end 103b. In one embodiment, the first channel 120 is open at both the upstream end 103a and downstream end 103b of the assembly 110 so that the guide wire 104 extends between the upstream and downstream ends 103a, 103b and additionally extends beyond the upstream and downstream ends 103a, 103b. Although FIGS. 1A and 1E do not depict the guide wire 104 extending beyond the downstream end 103b, in one embodiment the guide 104 extends through the opening 130 in the balloon 112 and beyond the downstream end 103b.

As depicted in FIG. 1D, in one embodiment, the system 100 includes an inlet port 102 positioned at the upstream end 103a and defines an end of the second channel 122 at the upstream end 103a. In an embodiment, fluid (e.g. saline contrast) is injected into the balloon lumen 105 at the inlet port 102 using an injection device (e.g. syringe). In an embodiment, the second channel 122 is open at the upstream end 103a (e.g. inlet port 102) of the assembly 110 and is closed at the downstream end 103b (e.g. within the balloon 112) of the assembly 110, in contrast with the first channel 120 that is open at both the upstream and downstream ends 103a, 103b.

As further depicted in FIG. 1D, in another embodiment, the system 100 includes a handle 106 that is operatively coupled to the deployment mechanism (e.g. deployment sleeve 114) so that movement of the handle 106 is configured to activate the deployment mechanism. In an example embodiment, retraction of the handle 106 retracts the deployment sleeve 114 relative to the stent 113 and thus causes the stent 113 to deploy. In another example embodiment, where the deployment mechanism is webbing that encloses the stent 113, the handle 106 includes a string that unravels the webbing and deploys the stent 113 when the string is pulled. In an example embodiment, the handle 106 features a pulley system with a cable. In another embodiment, the deployment mechanism is a trigger that retracts the deployment mechanism of the sleeve by an incremental length (e.g. 1 mm) by each click or activation of the trigger. In another embodiment, the deployment mechanism is a tuning dial that turns and rotates to retract the deployment mechanism with a perpendicular force. In an example embodiment, such a deployment mechanism is provided by a SMART® Control Stent System by Cordis®, Baar, Switzerland.

As depicted in FIG. 1D, in one embodiment, the balloon 112 is spaced apart from the downstream end 140 of the stent 113 by a minimum gap 302. In one embodiment, the minimum gap is about 5 mm or in a range from about 2 mm to about 10 mm. In another example embodiment, the minimum gap is in a range from about 1 mm to about 20 mm. In yet another example embodiment, the minimum gap is scaled based on the scale of the radiological display to ensure that the spacing between the balloon 112 and the downstream end 140 is discernible with the naked eye.

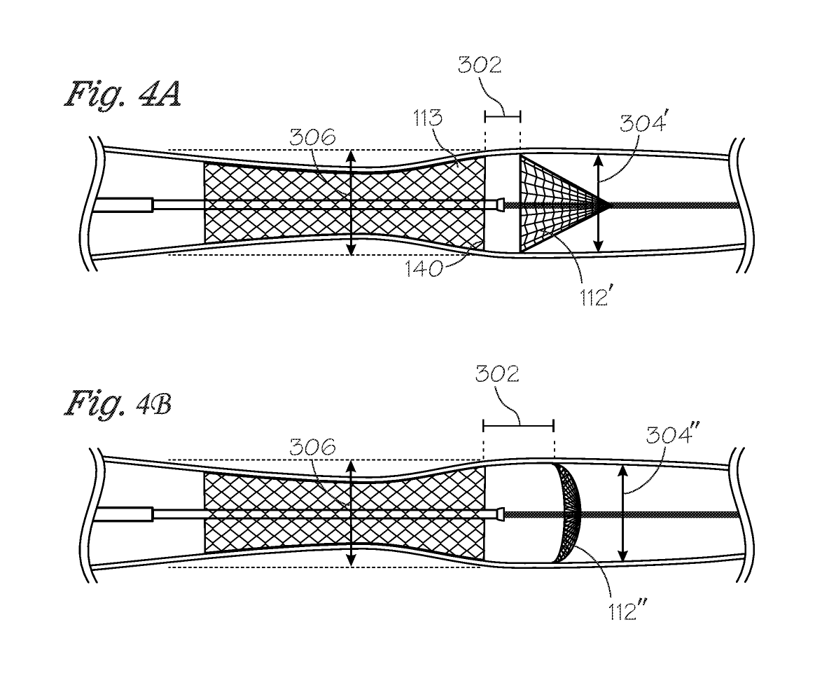

In another embodiment, the safety mechanism is a basket 112′ (FIG. 4A) that is also positioned on the downstream side of the stent 113, i.e. positioned downstream of the end 140 of the stent 113. In an example embodiment, the basket 112′ takes the form of an inferior vena cava (IVC) filter, as appreciated by one skilled in the art. In yet another embodiment, the safety mechanism is an umbrella 112″ (FIG. 4B) that is also positioned on the downstream side of the stent 113, i.e. positioned downstream of the end 140 of the stent 113. In an example embodiment, the basket 112′ or the umbrella 112′ are made from metallic material. In another example embodiment, a deployment mechanism for either the basket 112′ or the umbrella 112′ is received along the second channel 122 in lieu of the balloon lumen 105. In an example embodiment, the deployment mechanism for either the basket 112′ or the umbrella 112″ includes a wire or similar mechanism that is moved back and forth within the second channel 122 to open or close the basket 112′ or umbrella 112″.

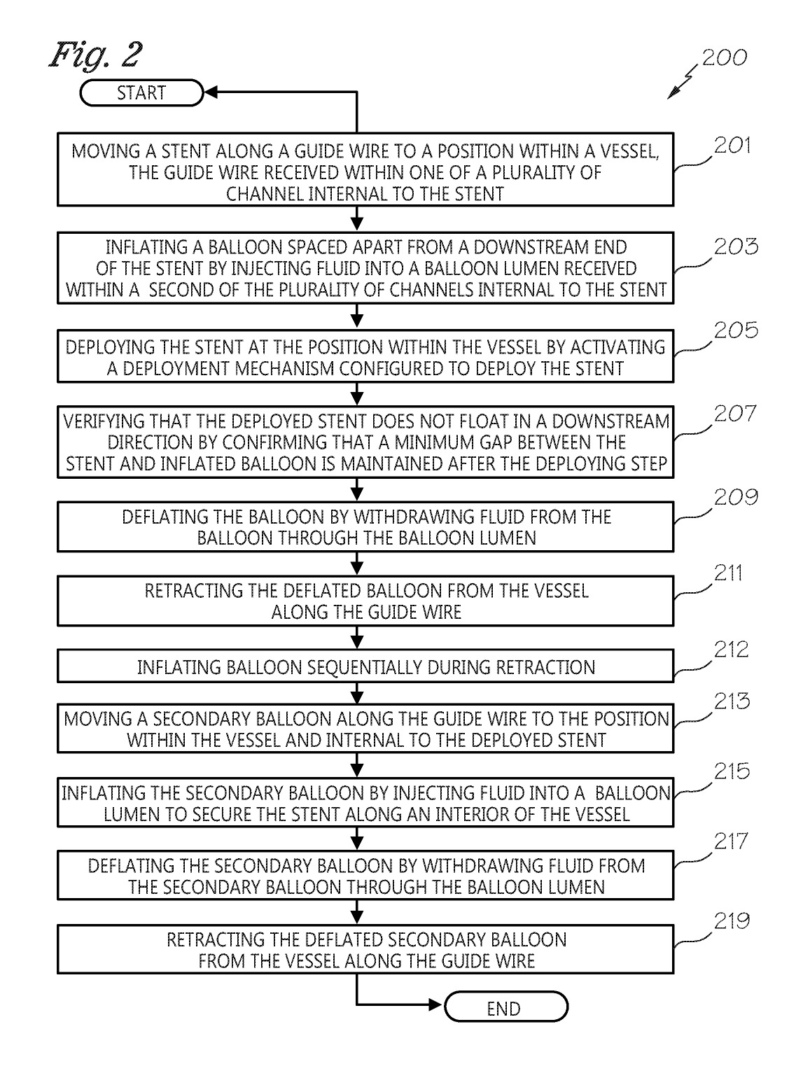

Although steps are depicted in the flowchart of FIG. 2 as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

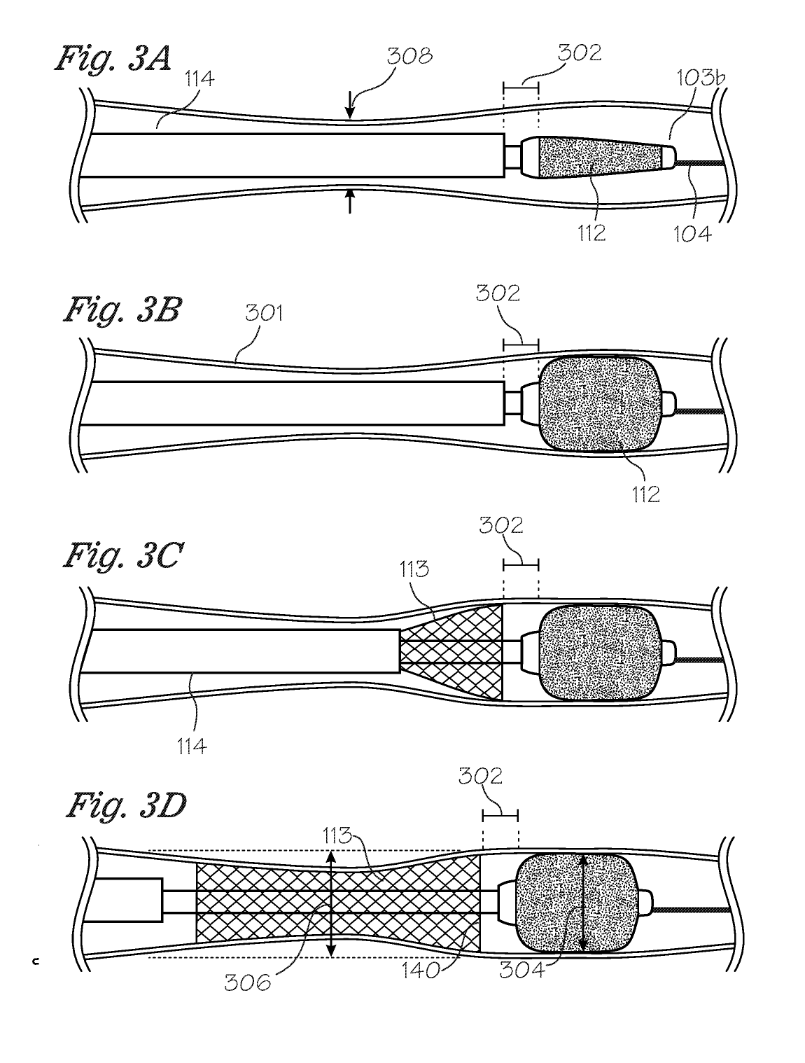

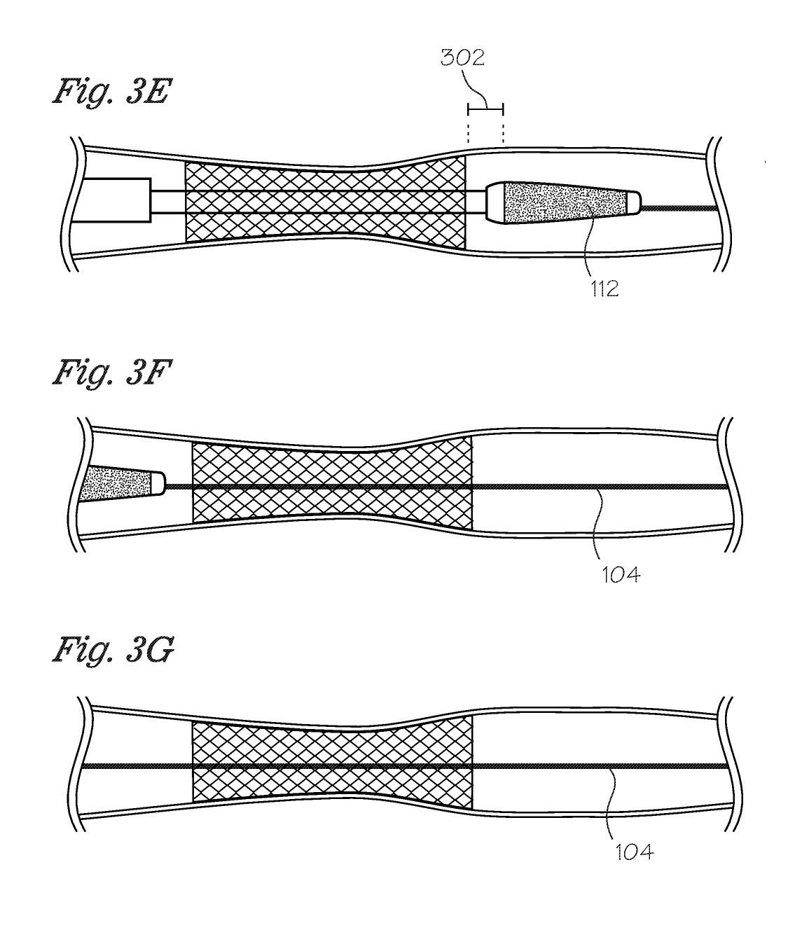

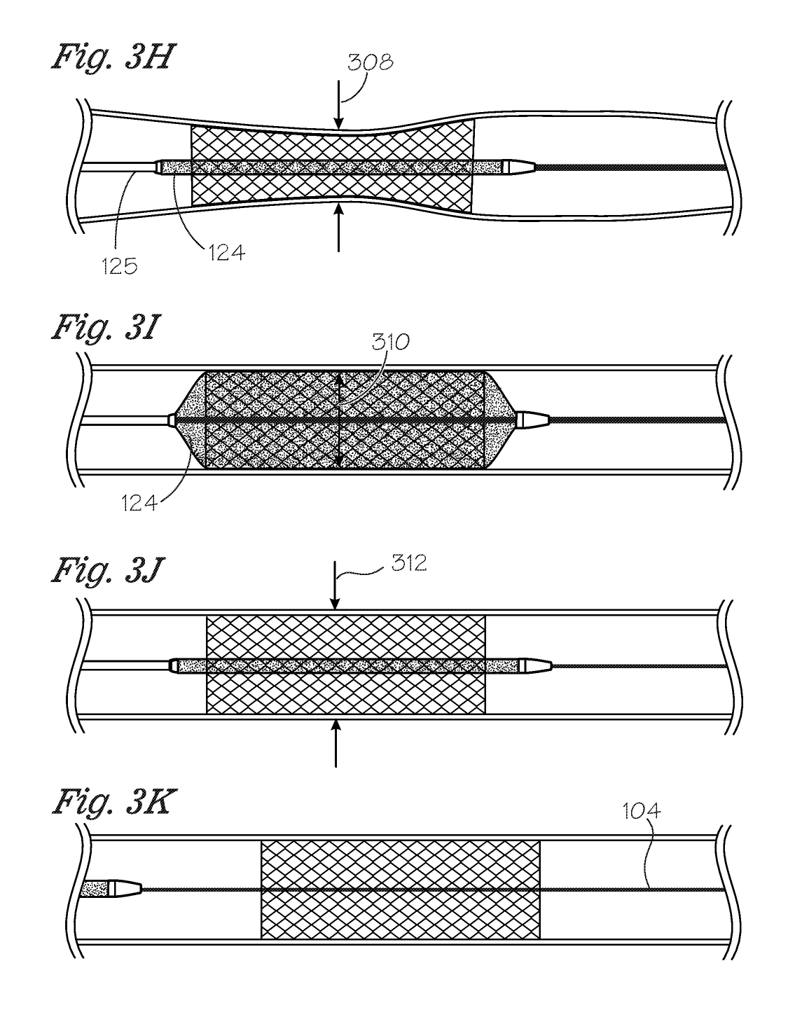

FIG. 2 is a flow chart that illustrates an example method 200 for deploying the stent 113 in a vessel 301 of a subject, according to an embodiment. In step 201, the stent 113 is moved along the guide wire 104 within the vessel 301. FIG. 3A is a side view that illustrates one example embodiment of this step. In an embodiment, in step 201, the guide wire 104 is initially positioned within the vessel 301. The assembly 110 is then slidably received over the guide wire 104 by slidably receiving the guide wire 104 in the first channel 120. In an embodiment, in step 301 the assembly 110 is moved over the guide wire 104 until the stent 113 is located at a position along the vessel 301 with a narrowed diameter 308. In an example embodiment, the narrowed diameter 308 is less than a deployment diameter 306 (FIG. 3D) of the stent 113 so that upon deployment the stent 113 secures along an inner surface of the vessel 301 at the narrowed diameter 308. In an example embodiment, during step 301 a medical professional (e.g. physician) moves the handle 106 in order to move the stent 113 along the vessel 301. In a further example embodiment, during step 301, the medical professional observes a real-time radiological display (e.g. X-ray) of the stent 113 moving along the vessel 301 until the medical professional observes that the stent 113 is located at the position with the narrowed diameter 308. In an example embodiment, the vessel 301 is one of an arterial vessel or a venous vessel and for each respective vessel 301 the narrowed diameter 308 is in a range from about 0 mm to about 20 mm. In an example embodiment, where the vessel 301 is closed (i.e. narrowed diameter 308 is about 0 mm) the guide wire 104 is first passed through the closed diameter to form an opening in the narrowed diameter 308 after which a balloon (e.g. secondary balloon 124) is passed along the guide wire 104 into the narrowed diameter 308 and is inflated to widen the diameter 308. After the diameter 308 has been widened, step 201 is performed. Although the vessel 301 is depicted in FIG. 3A, the vessel 301 is not a component of the system 100 or assembly 110. Additionally, in some embodiments, the assembly 110 or system 100 is used on vessels 301 of human subjects whereas in other embodiments, the assembly 110 or system 100 is used on vessels 301 of non-human subjects.

In step 203, the balloon 112 that is spaced apart from the downstream end 140 of the stent 113 is inflated. FIG. 3B is a side view that illustrates one example embodiment of this step. In one embodiment, in step 203 fluid (e.g. saline contrast) is injected into the inlet port 102 and thus into the balloon lumen 105. The injected fluid passes through the balloon lumen 105 and into the balloon 112 interior, causing the balloon 112 to inflate. In some embodiments, in step 203 the balloon 112 inflates and presses against the vessel 301 wall. In other embodiments, in step 203 the balloon 112 inflates and does not press against the vessel 301 wall. In an example embodiment, even if the balloon 112 inflates and does not press against the vessel 301 wall, the position of the balloon 112 along the vessel 301 remains fixed based on a medical professional holding the upstream end 103a of the assembly 110 in a fixed position.

In step 205, the stent 113 is deployed at the position in the vessel 301 with the narrowed diameter 308 by activating the deployment mechanism. FIG. 3C is a side view that illustrates one example embodiment of this step. In one embodiment, where the deployment mechanism is the deployment sleeve 114, in step 205 the medical professional (e.g. doctor) retracts the handle 106 which in turn causes the deployment sleeve 114 to retract relative to the stent 113 and the stent 113 to deploy. In an example embodiment, the stent 113 is spring-loaded and automatically deploys upon retraction of the deployment sleeve 114. In another embodiment, where the deployment mechanism is webbing that encloses the stent 113, in step 205 the medical professional (e.g. doctor) pulls a string that causes the webbing to unravel and the stent 113 to deploy. In an example embodiment, the stent 113 is spring-loaded and automatically deploys upon unraveling of the webbing.

In step 207, it is verified that the deployed stent 113 does not float in a downstream direction after the stent 113 is deployed. FIG. 3D is a side view that illustrates one example embodiment of this step. In one embodiment, in step 207 the verification that the deployed stent 113 has not floated in the downstream direction is performed by confirming that the minimum gap 302 between the stent 113 and the inflated balloon 112 is maintained after deployment of the stent 113. In an embodiment, the medical professional (e.g. physician) observes the real-time radiological display (e.g. X-ray) and visually confirms that the minimum gap 302 is maintained after deployment of the stent 113. In an example embodiment, step 207 is performed over a minimum time period to ensure that the minimum gap 302 is maintained. In one example embodiment, the minimum time period is in a range from about 1 second to about 3 seconds. In another example embodiment, the minimum time period is in a range from about 1 second to about 20 seconds.

In another embodiment, in step 207 the verification that the deployed stent 113 has not floated in the downstream direction involves configuring the balloon 112 so that an inflated diameter 304 of the balloon 112 is greater than a deployment diameter 306 of the stent 113. In an example embodiment, the deployment diameter 306 of the stent 113 is in a range from about 1 mm to about 20 mm for various stents used in various vessels and the inflated diameter 304 of the balloon 112 is in a range from about 2 mm to about 21 mm for a range of balloons used with these various stents. Thus, even if the stent 113 floats in the downstream direction towards the balloon 112, the balloon 112 advantageously acts as a physical barrier to prevent the stent 113 from floating beyond the balloon 112. In an example, the balloon 112 is made of Kevlar® material so that contact with the stent 113 will not cause the balloon 112 to deflate. In an embodiment, during step 207 the balloon 112 remains fixed within the vessel 301 based on the inflated balloon 112 pressing against the vessel 301 wall or the medical staff member holding the upstream end 103a or both. In an example embodiment, in the event the stent 113 floats in the direction of the balloon 112, the medical staff attempts to reposition the stent 113 back at the narrowed opening 308 in the vessel 301 or contacts emergency medical services if such repositioning is not successful. In the event that the stent 113 requires retrieval from the subject, the balloon 112 advantageously acts as a physical barrier to prevent the stent 113 from floating beyond the balloon 112 and thus greatly reduces the health risks to the subject as compared to if no physical barrier were present.

In step 209, the balloon 112 is deflated after performing the verification in step 207. FIG. 3E is a side view that illustrates one example embodiment of this step. In one embodiment, in step 209 the balloon 112 is deflated by withdrawing fluid from the balloon 112 through the balloon lumen 105. In an example embodiment, the fluid is withdrawn from the balloon lumen 105 at the inlet port 102.

In step 211, the deflated balloon 112 is retracted from the vessel 301. FIG. 3F is a side view that illustrates one example embodiment of this step. In one embodiment, in step 211 the balloon 112 is retracted along the guide wire 104 from the vessel 301 by sliding the balloon 112 along the guide wire 104 and out of the vessel 301. In an example embodiment, in step 211, a medical professional pulls the upstream end 103a and handle 106, causing the balloon 112 to slide along the guide wire 104 and out of the vessel 301. In another embodiment, in step 211, the balloon 112, the balloon lumen 105 and the deployment sleeve 114 are also retracted along the guide wire 104 from the vessel 301. FIG. 3G is a side view that illustrates one example embodiment of the vessel 301 after step 211, where only the guide wire 104 and deployed stent 113 remain positioned in the vessel 301.

In step 213, a secondary balloon 124 and balloon lumen 125 are moved along the guide wire 104 within the vessel 301. FIG. 3H is a side view that illustrates one example embodiment of this step. In one embodiment, in step 213 the secondary balloon 124 is moved to the position within the vessel 301 with the narrowed diameter 308 and/or to the position within the deployed stent 113. In an example embodiment, the secondary balloon 124 has a length that is longer than a length of the stent 113. In another example embodiment, the secondary balloon 124 has a length that is shorter than a length of the stent 113. In another example embodiment, the secondary balloon 124 is a non-compliant balloon that has a maximum pressure of about 20 atmospheres (atm) or in a range from about 2 atm to about 50 atm.

In step 215, the secondary balloon 124 is inflated. FIG. 3I is a side view that illustrates one example embodiment of this step. In an embodiment, in step 215, fluid (e.g. saline contrast) is injected into the balloon lumen 125 at the inlet port 102 using an injection device (e.g. syringe). In an embodiment, the inflated diameter 310 of the secondary balloon 124 is greater than the narrowed diameter 308 (FIG. 3H) of the vessel 301 so that step 215 causes the narrowed diameter 308 of the vessel 301 to increase to an opening diameter 312 (FIG. 3J). In an example embodiment, step 215 advantageously widens the diameter of the vessel 310. In another embodiment, inflation of the secondary balloon 124 in step 215 further secures the deployed stent 113 along an interior wall of the vessel 301 so that the stent 113 is permanently secured to the vessel 301 at the deployed position.

In step 217, the secondary balloon 124 is deflated. FIG. 3J is a side view that illustrates one example embodiment of this step. In one embodiment, in step 217 the secondary balloon 124 is deflated by withdrawing fluid from the balloon 124 through the balloon lumen 125. In an example embodiment, the fluid is withdrawn from the balloon lumen 125 at the inlet port 102. As depicted in FIG. 3J the opening diameter 312 has increased from the narrowed diameter 308 prior to steps 215 and 217 to the opening diameter 312.

In step 219, the secondary balloon 124 and balloon lumen 125 are retracted from the vessel 301. FIG. 3K is a side view that illustrates one example embodiment of this step. In one embodiment, in step 219 the balloon 124 is retracted along the guide wire 104 from the vessel 301 by sliding the balloon 124 along the guide wire 104 and out of the vessel 301. In an example embodiment, in step 219, a medical professional pulls the upstream end 103a and handle 106, causing the balloon 124 to slide along the guide wire 104 and out of the vessel 301. In another embodiment, in step 219, the balloon 124 and the balloon lumen 125 are retracted along the guide wire 104. In some embodiments, in a subsequent step after step 219, the guide wire 104 is removed from the vessel 301.

In some embodiments of the method 200, steps 211, 215, 217, 219 are modified and step 213 is omitted. In these embodiments, in step 211 the deflated balloon 112 is not retracted from the vessel 301 along the guide wire 104 and instead the deflated balloon 112 is moved along the guide wire 104 to the position within the deployed stent 113 (e.g. the same position as the secondary balloon 124 in FIG. 3H) or the position with the narrowed diameter 308. In an example embodiment, this embodiment involves step 212 of inflating the balloon 112 sequentially during the retraction step 211. In these embodiments, steps 215, 217, 219 are performed using the balloon 112 instead of the secondary balloon 124. Thus, in step 215, the balloon 112 is inflated after positioning the balloon 112 at the position within the deployed stent 113. In an example embodiment, in step 203, the balloon 112 is inflated to a low pressure by injecting fluid at a reduced pressure into the balloon lumen 105 at the inlet port 102. In an example embodiment, the material of the balloon 112 (e.g. Kevlar®) is selected such that the injected fluid at the reduced pressure results in the balloon 112 being inflated to the desired low pressure. In an example embodiment, after retracting the balloon 112 to a location within the deployed stent 113, in step 215 the balloon 112 is inflated to a high pressure (greater than the low pressure in step 203) within the stent 113 by injecting fluid at a greater pressure (that exceeds the reduced pressure in step 203) into the balloon lumen 105 at the inlet port 102 so that the inflated balloon 112 presses the deployed stent 113 into the interior walls of the vessel 301 and thus further secures the deployed stent 113 to the vessel 301. In an example embodiment, the material of the balloon 112 (e.g. Kevlar®) is selected such that the injected fluid at the greater pressure results in the balloon 112 being inflated to the desired high pressure. In another example embodiment, the balloon 112 is inflated such that the diameter of the vessel 301 increases from the narrowed diameter 308 to the opening diameter 312 depicted in FIG. 3J. In an example embodiment, the balloon 112 has a maximum pressure in a range of about 5 atm to about 25 atm. In step 217, the balloon 112 is deflated by withdrawing fluid along the balloon lumen 105 at the inlet port 102. In step 219, the balloon 112 is retracted along the guide wire 104 from the vessel 301.

In one embodiment, the method 200 can be performed using the basket 112′ (FIG. 4A) in lieu of the balloon 112. The steps of this embodiment are similar to the steps of the method 200 using the balloon 112, with the exception that the basket 112′ is used in place of the balloon 112 and a deployment mechanism of the basket 112′ is used to deploy the basket 112′ in lieu of the balloon lumen 105. In an example embodiment, the deployment mechanism of the basket 112′ is positioned along the second channel 122 in lieu of the balloon lumen 105. As depicted in FIG. 4A, the deployment diameter 304′ of the deployed basket 112′ is greater than the deployment diameter 306 of the stent 113 such that upon deployment of the basket 112′ and subsequent deployment of the stent 113, the deployment diameter 304′ of the basket 112′ provides a physical barrier to prevent the deployed stent 113 from floating or migrating in a downstream direction beyond the basket 112′.

In one embodiment, the method 200 can be performed using the umbrella 112″ (FIG. 4B) in lieu of the balloon 112. The steps of this embodiment are similar to the steps of the method 200 using the balloon 112, with the exception that the umbrella 112″ is used in place of the balloon 112 and a deployment mechanism of the umbrella 112″ is used to deploy the umbrella 112″ in lieu of the balloon lumen 105. In an example embodiment, the deployment mechanism of the umbrella 112″ is positioned along the second channel 122 in lieu of the balloon lumen 105. As depicted in FIG. 4B, the deployment diameter 304″ of the deployed umbrella 112″ is greater than the deployment diameter 306 of the stent 113 such that upon deployment of the umbrella 112″ and subsequent deployment of the stent 113, the deployment diameter 304″ of the umbrella 112″ provides a physical barrier to prevent the deployed stent 113 from floating or migrating in a downstream direction beyond the umbrella 112″.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article. As used herein, unless otherwise clear from the context, a value is “about” another value if it is within a factor of two (twice or half) of the other value. While example ranges are given, unless otherwise clear from the context, any contained ranges are also intended in various embodiments. Thus, a range from 0 to 10 includes the range 1 to 4 in some embodiments.

1. An assembly comprising:
a stent;
a plurality of channels positioned internal to the stent; and
a deployment mechanism configured to deploy the stent when the stent is positioned in a vessel of a subject.

2. An assembly as claimed in claim 1, wherein the deployment mechanism is a deployment sleeve that is configured to deploy the stent upon retraction of the deployment sleeve relative to the stent.

3. An assembly as claimed in claim 1, wherein the deployment mechanism comprises webbing enclosing the stent and a string configured to unravel the webbing upon pulling the string.

4. An assembly as claimed in claim 1, further comprising a safety mechanism positioned distally to the stent, said safety mechanism configured to obstruct motion of the deployed stent migrating in the vessel.

5. An assembly as claimed in claim 4, wherein the safety mechanism is a basket positioned on a downstream side of the stent.

6. An assembly as claimed in claim 5, wherein a deployment diameter of the basket is greater than a deployment diameter of the stent such that upon deployment of the basket and subsequent deployment of the stent, the deployment diameter of the basket being greater than the deployment diameter of the stent provides a physical barrier to prevent the deployed stent floating in a downstream direction beyond the basket.

7. An assembly as claimed in claim 4, wherein the safety mechanism is an umbrella positioned on a downstream side of the stent.

8. An assembly as claimed in claim 7, wherein a deployment diameter of the umbrella is greater than a deployment diameter of the stent such that upon deployment of the umbrella and subsequent deployment of the stent, the deployment diameter of the umbrella being greater than the deployment diameter of the stent provides a physical barrier to prevent the deployed stent floating in a downstream direction beyond the basket.

9. An assembly as claimed in claim 1, further comprising:
a balloon positioned on a downstream side of the stent;
a guide wire;
a balloon lumen; and
wherein the plurality of channels comprises a first channel configured to receive the guide wire and a second channel configured to receive the balloon lumen.

10. An assembly as claimed in claim 9, wherein the first channel is open at an upstream end and a downstream end of the assembly such that the guide wire extends between the upstream end and the downstream end of the assembly.

11. An assembly as in claim 10, wherein the guide wire extends beyond the upstream end and the downstream end and wherein the first channel extends through an opening formed by the balloon.

12. An assembly as in claim 9, wherein the second channel is open at an upstream end of the assembly and is closed at a downstream end of the assembly.

13. An assembly as in claim 12, wherein the second channel is open at the upstream end of the assembly to inject fluid into the balloon lumen at the upstream end of the assembly and wherein the second channel is closed at a downstream end of the assembly based on a downstream end of the balloon lumen being positioned within an interior of the balloon to direct the injected fluid into the interior of the balloon to inflate the balloon.

14. An assembly as in claim 9, wherein the balloon is spaced apart from a downstream end of the stent by a minimum gap such that upon inflation of the balloon and subsequently deployment of the stent, the minimum gap provides visual confirmation that the deployed stent has not floated in a downstream direction relative to the inflated balloon.

15. An assembly as in claim 9, wherein an inflated diameter of the balloon is greater than a deployment diameter of the stent such that upon inflation of the balloon and subsequent deployment of the stent, the inflated diameter of the balloon being greater than the deployment diameter of the stent provides a physical barrier to prevent the deployed stent floating in a downstream direction beyond the inflated balloon.

16. An assembly as in claim 1, further comprising a balloon and a balloon lumen, wherein the balloon is positioned internal to the stent upon deployment of the stent and fluid is injected through the balloon lumen to inflate the balloon and secure the stent along an interior of the vessel.

17. An assembly as in claim 16, wherein the balloon is initially spaced apart from a downstream end of the stent prior to deployment of the stent and is inflated prior to deployment of the stent to ensure that the deployed stent does not float in a downstream direction and wherein the balloon is subsequently deflated and positioned internal to the stent prior to inflating the balloon to secure the stent along the interior of the vessel.

18. A method comprising:
moving a stent to a position along a vessel;
inflating a balloon positioned on a downstream side of the stent;
deploying the stent at the position;
verifying that the deployed stent does not float from the position in a downstream direction relative to the inflated balloon; and
deflating the balloon.

19. (canceled)

20. (canceled)

21. (canceled)

23. A method as in claim 18, wherein an inflated diameter of the balloon is greater than a deployment diameter of the stent and wherein the verifying step comprises the inflated balloon providing a physical barrier to the deployed stent from floating in the downstream direction beyond the inflated balloon.

24. (canceled)

25. (canceled)

26. (canceled)

27. (canceled)

28. (canceled)

29. (canceled)

30. An assembly comprising:
a stent;
a catheter positioned internal to the stent, the catheter comprising one or more channels;
a deployment mechanism configured to deploy the stent when the stent is positioned in a vessel of a subject;
a safety mechanism positioned distally to the stent, said safety mechanism configured to obstruct motion of the deployed stent migrating in the vessel.

31. (canceled)

32. (canceled)
